(19) United States
(12) Patent Application Publication
(10) Pub. No.: US 2020/0176097 A1
(43) Pub. Date: Jun. 4, 2020

(54) DISPLAY DEVICE AND DISPLAY METHOD

(71) Applicant: KYOCERA Document Solutions Inc., Osaka (JP)

(72) Inventor: Haruo ITO, Osaka-shi (JP)

(73) Assignee: KYOCERA Document Solutions Inc., Osaka (JP)

(21) Appl. No.: 16/695,516

(22) Filed: Nov. 26, 2019

(30) Foreign Application Priority Data
Nov. 29, 2018 (JP) 2018-223302

(51) Int. Cl. G16H 15/00; G06K 9/00; G06F 3/14; G16H 10/60; G16H 20/60; G16H 50/30

(52) U.S. Cl. CPC G16H 15/00; G06K 9/00442; G06F 3/14; G06K 2209/01; G16H 20/60; G16H 50/30; G16H 10/60

(57) ABSTRACT

A display device includes an acquiring section, a specifying section, an estimating section, and a display section. The acquiring section acquires health data on health. The specifying section specifies effect data that has effect on the health data. The estimating section estimates an increase or decrease in the health data based on the effect data to obtain an estimated result. The display section displays the estimated result.

### INCORPORATION BY REFERENCE

The present application claims priority under 35 U.S.C. § 119 to Japanese Patent Application No. 2018-223302, filed on Nov. 29, 2018. The contents of this application are incorporated herein by reference in their entirety.

### BACKGROUND

The present disclosure relates to a display device, and a display method.

Devices that cause a computer(s) to manage collection and analysis of information about user health have been examined.

In a technique, based on analysis information registered in advance, health condition information indicating measurement results related to health is analyzed, and the analysis result is output.

### SUMMARY

A display device according to an aspect of the present disclosure includes an acquiring section, a specifying section, an estimating section, and a display section. The acquiring section acquires health data on health. The specifying section specifies effect data that has effect on the health data. The estimating section estimates an increase or decrease in the health data based on the effect data to obtain an estimated result. The display section displays the estimated result.

A display method according to an aspect of the present disclosure includes acquiring health data on health, specifying effect data that has effect on the health data, estimating a variation of the health data based on the effect data to obtain an estimated result, and displaying the estimated result.

### BRIEF DESCRIPTION OF THE DRAWINGS

FIG. 1 is a schematic diagram depicting a display system according to a first embodiment of the present disclosure.

FIG. 2 is a block diagram depicting a configuration of a display device in the display system.

FIG. 3 illustrates a function of the display device.

FIG. 4A depicts an example of a user information table storing user information. FIG. 4B depicts an example of a health data table storing heath data.

FIG. 5 depicts an example of an effect data table storing effect data.

FIG. 6 depicts an example of an estimation data table storing estimation data.

FIG. 7 depicts an example of an estimated result table storing estimated results.

FIG. 8 is a communication sequence diagram of the display system according to the first embodiment.

FIG. 9 is a schematic diagram depicting a display system according to a second embodiment of the present disclosure.

### DETAILED DESCRIPTION

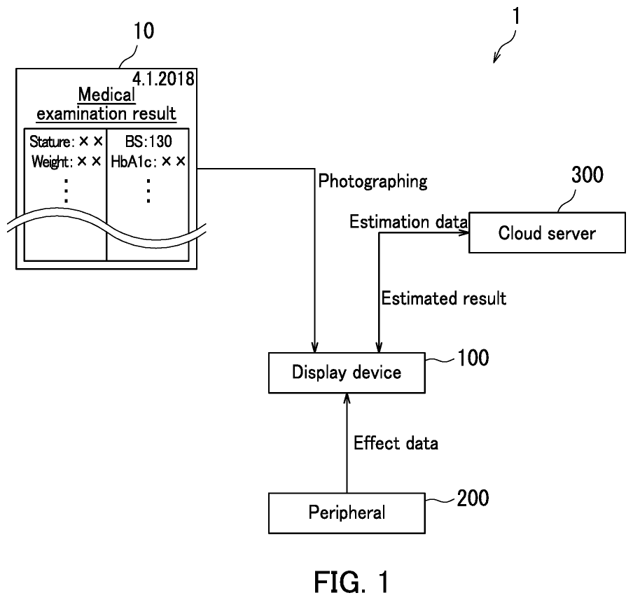

Embodiments of the present disclosure will hereinafter be described with reference to the accompanying drawings (FIGS. 1 to 9). Note that elements that are the same or equivalent are labelled with the same reference signs in the drawings and description thereof is not repeated.

### First Embodiment

FIG. 1 is a schematic diagram depicting a display system 1 according to a first embodiment of the present disclosure. The display system 1 is a system that supports user health promotion activities related to a medical examination result 10. As illustrated in FIG. 1, the display system 1 includes a display device 100, a peripheral 200, and a cloud server 300. Note that the medical examination result 10 is an example of a “report”.

The display device 100 displays an image. The display device 100 further transmits external light. The display device 100 is for example a transparent (see-through) liquid-crystal display that displays a color image.

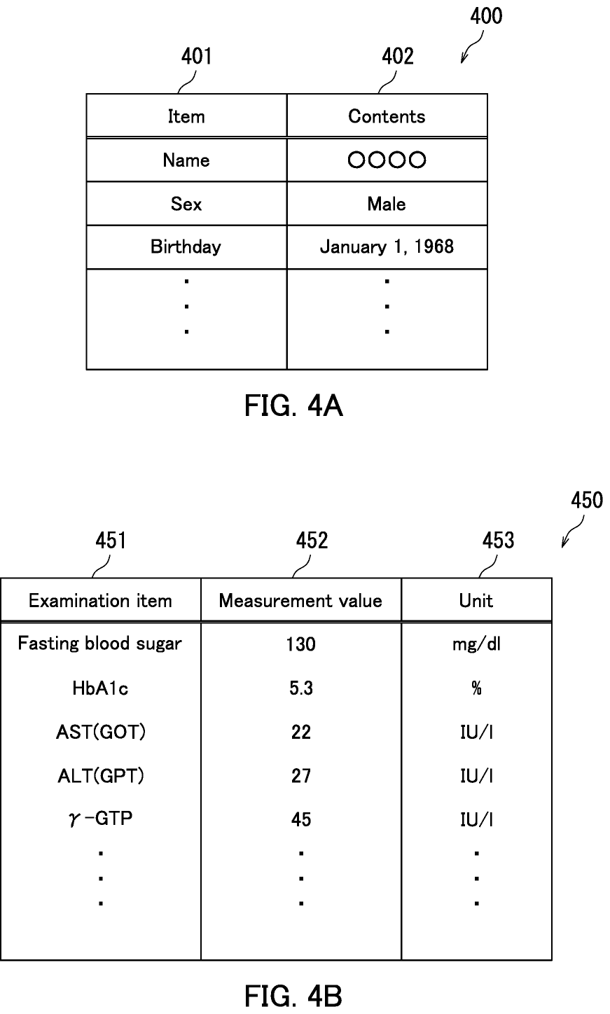

In the present embodiment, the display device 100 is equipped with a built-in camera, and has an optical character recognition (OCR) function. The display device 100 photographs the medical examination result 10 to capture health data indicating results of the medical examination. Note that the health data will be described in detail with reference to FIG. 4B.

The peripheral 200 collects effect data that has effect (influence) on the health data. Specifically, the peripheral 200 collects various pieces of effect data that improves or damages user health to be transmitted to the display device 100. The peripheral 200 generates a signal indicating whether a light in a bedroom of a user is turned on or off in a period from turning off the light to getting up. Note that the peripheral 200 is an example of a “specifying section”.

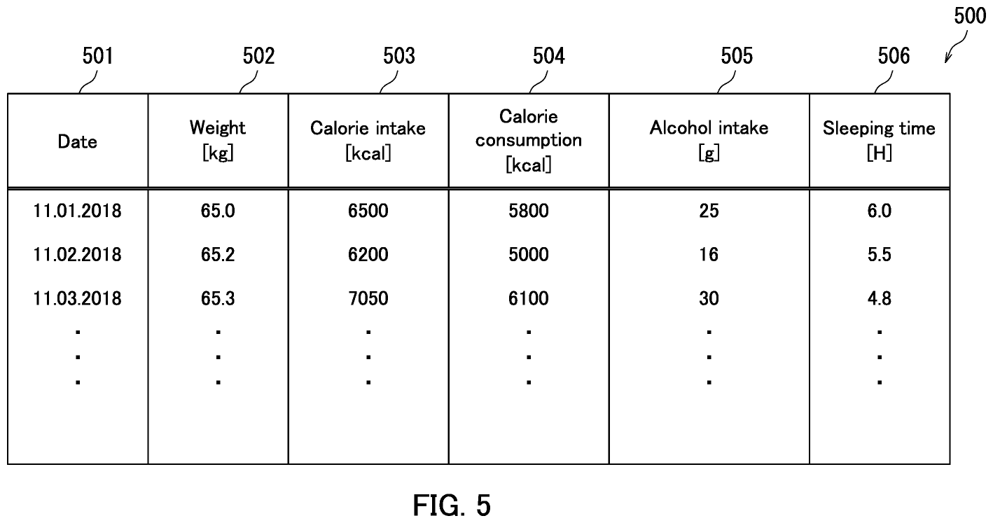

Examples of the effect data include daily body weight, calorie intake every one or more days as unit of time, calorie consumption every one or more days as unit of time, alcohol intake every one or more days as unit of time, and sleeping time every one or more days as unit of time. Here, the “one or more days as unit of time” is one or more days as a reference, during which the effect data is collected. In the present embodiment, the one or more days as unit of time are for example “one day”. Note that the effect data will be described in detail with reference to FIG. 5.

The display device 100 receives the effect data from the peripheral 200. The display device 100 also specifies estimation data based on the effect data. Here, the “estimation data” is data to be used for estimating an increase or decrease in the health data.

The display device 100 transmits the estimation data to the cloud server 300 to make a request for estimation about the increase or decrease in the health data. The display device 100 further receives (an) estimated results from the cloud server 300. The display device 100 then displays the estimated results received from the cloud server 300. Here, the “estimated results” are results obtained by performing estimation about the increase or decrease in the health data.

The cloud server 300 estimates the increase or decrease in the health data based on the estimation data received from the display device 100. The cloud server 300 further transmits, to the display device 100, the estimated results obtained by performing the estimation about the increase or decrease in the health data. Also, based on position information received from the display device 100, the cloud server 300 may determine whether or not the place indicated by the position information is a restaurant. When determining that the place is the restaurant, the cloud server 300 may estimate calories of the meal.

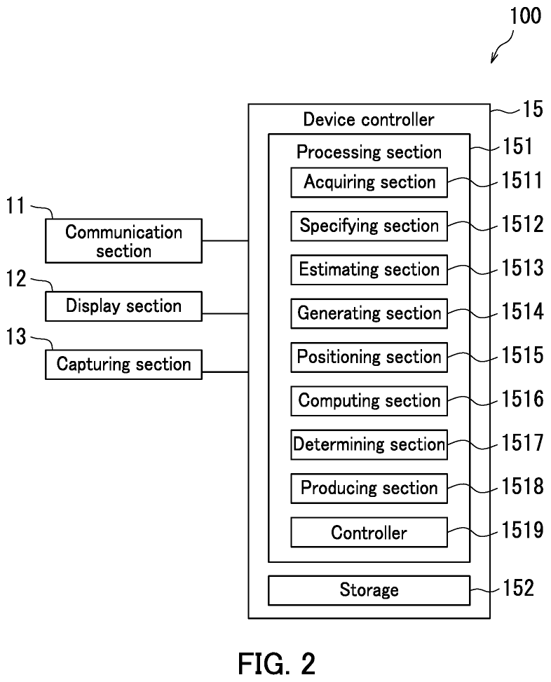

A configuration of the display device 100 will next be described with reference to FIG. 2. FIG. 2 is a block diagram depicting the configuration of the display device 100. As illustrated in FIG. 2, the display device 100 includes a communication section 11, a display section 12, a capturing section 13, and a device controller 15.

The communication section 11 transmits and receives various pieces of data to and from a different electronic device in accordance with an instruction of the device controller 15. Specifically, the device controller 15 receives effect data from the peripheral 200, examples of which include a smartwatch. The communication section 11 is for example a communication interface.

The communication section 11 further transmits estimation data specified by the device controller 15 to the cloud server 300. The communication section 11 may transmit position information indicating a position of the display device 100 to the cloud server 300. The communication section 11 also receives the estimated results from the cloud server 300. Note that the communication section 11 is an example of a “detecting section”.

The communication section 11 receives, from the peripheral 200, a signal indicating whether the light in the bedroom of the user is turned on or off in the period from turning off the light to getting up.

The display section 12 displays the estimated results received from the cloud server 300 through the communication section 11. Examples of the display section 12 include augmented reality (AR) glasses including a pair of left and right glasses, and a head mounted display (HMD). In the present embodiment, the display section 12 projects or displays an estimated image generated by the device controller 15. The display section 12 includes a transparent liquid-crystal display that displays a color image. Note that the display section 12 is not limited to the transparent liquid-crystal display. The display section 12 may include an organic electroluminescent (EL) display.

The capturing section 13 photographs the medical examination result 10 containing the health data to generate image data representing an image of the medical examination result 10. The capturing section 13 further photographs each meal to generate meal image data representing meal contents. Here, the meal contents may include beer or sake.

Examples of the capturing section 13 include a charge coupled device (CCD) image sensor, and a complementary metal oxide semiconductor (CMOS) image sensor.

The device controller 15 controls respective operations of components constituting the display device 100 based on a control program. The device controller 15 includes a processing section 151, and storage 152. The processing section 151 is for example a processor. The processor is for example a central processing unit (CPU).

The processing section 151 executes the control program stored in the storage 152, thereby controlling the respective operations of the components constituting the display device 100.

The storage 152 stores various pieces of data and the control program. The storage 152 includes at least one of devices, examples of which include read-only memory (ROM), random-access memory (RAM), and a solid-state drive (SSD).

The processing section 151 includes an acquiring section 1511, a specifying section 1512, an estimating section 1513, a generating section 1514, a positioning section 1515, a computing section 1516, a determining section 1517, a producing section 1518, and a controller 1519. In the present embodiment, the processing section 151 executes the control program stored in the storage 152, thereby realizing respective functions of the acquiring section 1511, the specifying section 1512, the estimating 5-section 1513, the generating section 1514, the positioning section 1515, the computing section 1516, the determining section 1517, the producing section 1518, and the controller 1519.

The acquiring section 1511 acquires the health data on user health. Specifically, from the medical examination result 10, the acquiring section 1511 acquires, as the health data, at least one of values, examples of which include a numerical value indicating liver function, a numerical value indicating kidney function, a numeric value indicating lipid, and a numeric value indicating sugar.

In the present embodiment, the acquiring section 1511 includes the OCR function. The acquiring section 1511 uses the OCR function, thereby recognizing health data contained in the image of the medical examination result 10 from the image data generated by the capturing section 13.

The acquiring section 1511 also has a pedometer function. For example, the acquiring section 1511 acquires the number of steps in a period from getting up to going to bed.

The specifying section 1512 specifies the effect data that has effect on the health data. In the present embodiment, the specifying section 1512 specifies the effect data collected by the peripheral 200. Specifically, the specifying section 1512 specifies, as the effect data, calorie intake every one day as unit of time, calorie consumption every one day as unit of time, alcohol intake every one day as unit of time, and sleeping time every one day as unit of time.

The calorie intake every one day as unit of time is specified by the specifying section 1512 based on the calories of meals calculated by the computing section 1516. The alcohol intake every one day as unit of time is also specified by the specifying section 1512 based on the alcohol amount calculated by the computing section 1516.

The calorie intake every one day as unit of time is also specified by the specifying section 1512 based on the calories of meals estimated by the estimating section 1513.

The calorie consumption every one day as unit of time is also specified by the specifying section 1512 based on the calorie consumption calculated by the computing section 1516.

The sleeping time every one day as unit of time is also specified by the specifying section 1512 based on sleep time measured by the computing section 1516.

The specifying section 1512 also specifies, as estimation data based on the effect data, total calorie intake during a reference period, total calorie consumption during the reference period, total alcohol intake during the reference period, and average sleeping time during the reference period. Here, the “reference period” is a reference period during which the effect data is collected for estimating the increase or decrease in the health data. The reference period is for examples “seven days”.

The estimating section 1513 estimates the increase or decrease in the health data based on the effect data specified by the specifying section 1512 to obtain an estimated result. Specifically, the estimating section 1513 instructs the cloud server 300 to estimate the increase or decrease in the health data based on the effect data, and then obtains the estimated result from the cloud server 300. In the present embodiment, the estimating section 1513 causes the cloud server 300 to estimate the increase or decrease in the health data based on the effect data during the reference period.

The estimating section 1513 further causes the cloud server 300 to determine whether or not the place indicated by the position information is a restaurant based on the position information acquired by the positioning section 1515, and to estimate calories of the meal when it is determined that the place is the restaurant.

The generating section 1514 generates the estimated image representing the increase or decrease in the health data estimated by the estimating section 1513.

The positioning section 1515 acquires position information indicating the position of the display device 100. In the present embodiment, the positioning section 1515 has a global positioning system (GPS) function. The positioning section 1515 uses the GPS function, thereby generating the position information indicating the position of the display device 100.

The computing section 1516 computes calories of meals based on the meal image data generated by the capturing section 13. The computing section 1516 computes an alcohol amount based on the meal image data.

The computing section 1516 also computes calorie consumption based on the steps acquired by the acquiring section 1511.

The computing section 1516 also measures sleeping time in the period from turning off the light to getting up based on the signal detected through the communication section 11.

The determining section 1517 determines whether or not “a variation (a change in the amount) of the health data” is greater than a first threshold but not greater than a second threshold greater than the first threshold. Here, the “variation of the health data” refers to a difference between the health data of the medical examination result 10 and the health data estimated by the estimating section 1513.

The determining section 1517 further determines whether or not the variation of the health data is greater than the second threshold.

The producing section 1518 produces a prescribed message based on the determination result by the determining section 1517. Specifically, when the determining section 1517 determines that the variation of the health data is greater than the first threshold but not greater than the second threshold greater, the producing section 1518 produces a first message. For example, let a first threshold of “20” and a second threshold of “40” be set with respect to fasting blood sugar. In the case where the thresholds are set like this, the producing section 1518 produces, as the first message, for example a message saying, “Blood sugar level has returned to almost normal.” The producing section 1518 further produces a second message when the determining section 1517 determines that the variation of the health data is greater than the second threshold. The producing section 1518 produces, as the second message, for example a message saying, “Blood sugar level is in the normal range.”

The controller 1519 controls the display section 12. Specifically, the controller 1519 controls the display section 12 so that display is performed with the health data represented by the medical examination result 10 associated with the estimated results.

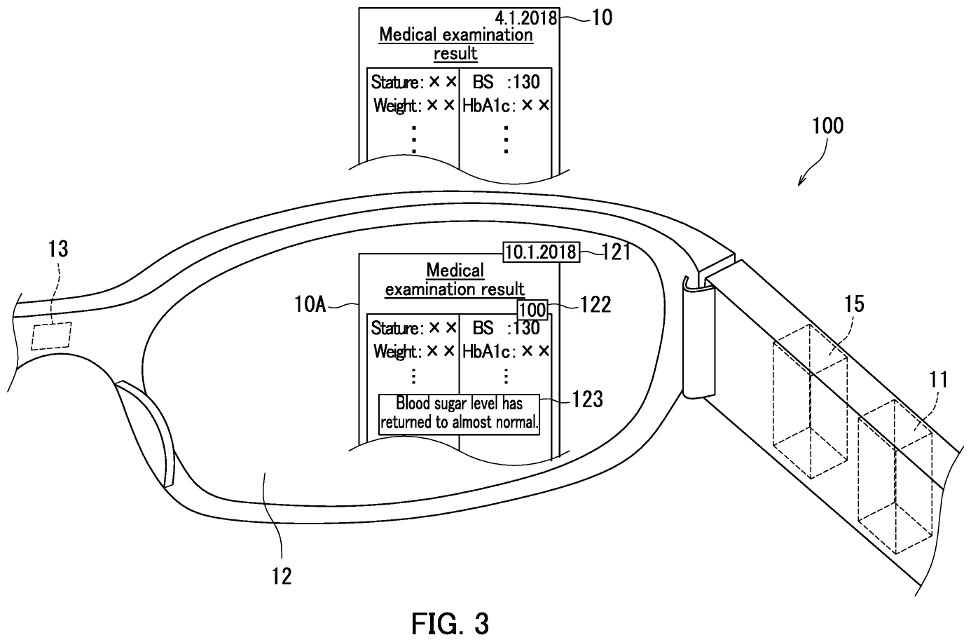

A function of the display device 100 will next be described with reference to FIGS. 2 and 3. FIG. 3 illustrates the function of the display device 100.

FIG. 3 depicts the display device 100 and the medical examination result 10. The display device 100 includes the communication section 11, the display section 12, the capturing section 13, and the device controller 15. The display section 12 is a transparent display, and allows a user to visually recognize the medical examination result 10. The display section 12 of the display device 100 displays a projection image 10A of the medical examination result 10. The display section 12 further displays an estimated image 122 of “100” generated by the device controller 15 in the vicinity of the fasting blood sugar level of “130” represented by the medical examination result 10. The display section 12 further displays a date image 121 representing date (Oct. 1, 2018), and a first message, generated by the device controller 15. As illustrated in FIG. 3, the controller 1519 controls the display section 12 so that the display by the display section 12 is performed with the date image 121 and the estimated image 122 associated with the date of the medical examination result 10 and health data, respectively. This enables the user to easily grasp the variation of the health data from the date of the medical examination to the date represented by the date image 121.

The controller 1519 further controls the display section 12 so that the first message produced by the producing section 1518 is displayed. The controller 1519 also controls the communication section 11 so that the second message produced by the producing section 1518 is transmitted to a prescribed electronic device. Here, the “prescribed electronic device” is for example a smartphone.

Various pieces of data stored in the storage 152 of the display device 100 will next be described with reference to FIGS. 4A to 6. FIG. 4A depicts an example of a user information table 400 storing user information. The “user information” refers to information that is used to specify a medical examinee indicated by the medical examination result 10. Note that the medical examinee of the medical examination result 10 is identical to a user of the display device 100 in the present embodiment.

The user information table 400 includes an item column (hereinafter simply called “items 401”) and a contents column (hereinafter simply called “contents 402”). The items 401 represent information items for specifying the medical examinee. Examples of the items 401 include name, sex, and birthday. The contents 402 represent specific information on each item in the items 401.

FIG. 4B depicts an example of a health data table 450 storing heath data. The heath data contains values, examples of which include a numerical value indicating liver function, a numerical value indicating kidney function, a numeric value indicating lipid, and a numeric value indicating sugar.

Examples of the numerical value indicating liver function include AST(GOT), ALT(GPT), γ-GTP, ALP, and total bilirubin. Examples of the numerical value indicating kidney function include creatinine, and uric acid. Examples of the numeric value indicating lipid include neutral fat, HDL-C, and LDL-C. Examples of the numeric value indicating sugar include fasting blood sugar, and HbA1c.

The health data table 450 represents measurement values or examination results about the examinee health. As illustrated in FIG. 4B, the health data table 450 includes an examination item column (hereinafter simply called “examination items 451”), a measurement value column (hereinafter simply called “measurement values 452”), and a unit column (hereinafter simply called “units 453”).

The examination items 451 are a column representing examination items. Examples of the examination items 451 include fasting blood sugar, and HbA1c. The measurement values 452 represent respective specific measurement results of the examination items 451. The units 453 represent respective reference amounts or forms when numerically expressing the examination items 451.

FIG. 5B depicts an example of an effect data table 500 storing effect data. The effect data table 500 represents effect data collected by the user on a daily basis. In the present embodiment, the effect data table 500 includes a date column (hereinafter simply called “dates 501”), a weight column (hereinafter simply called “weight 502”, a calorie intake column (hereinafter simply called “calorie intake 503”), a calorie consumption column (hereinafter simply called “calorie consumption 504”, an alcohol intake column (hereinafter simply called “alcohol intake 505”), and a sleeping time column (hereinafter simply called “sleeping time 506”). The dates 501 represent dates on which respective pieces of effect data are collected. The weight 502 represents body weight of the user collected on each date represented by the dates 501.

In the present embodiment, the calorie intake 503 represents total calorie intake of the user on each date. The calorie consumption 504 represents total calorie consumption of the user on each date. Each item of the calorie consumption 504 includes basal metabolism calories. The alcohol intake 505 represents total alcohol intake of the user on each date. The sleeping time 506 represents total sleeping time of the user from 0:00 to 24:00 on each date.

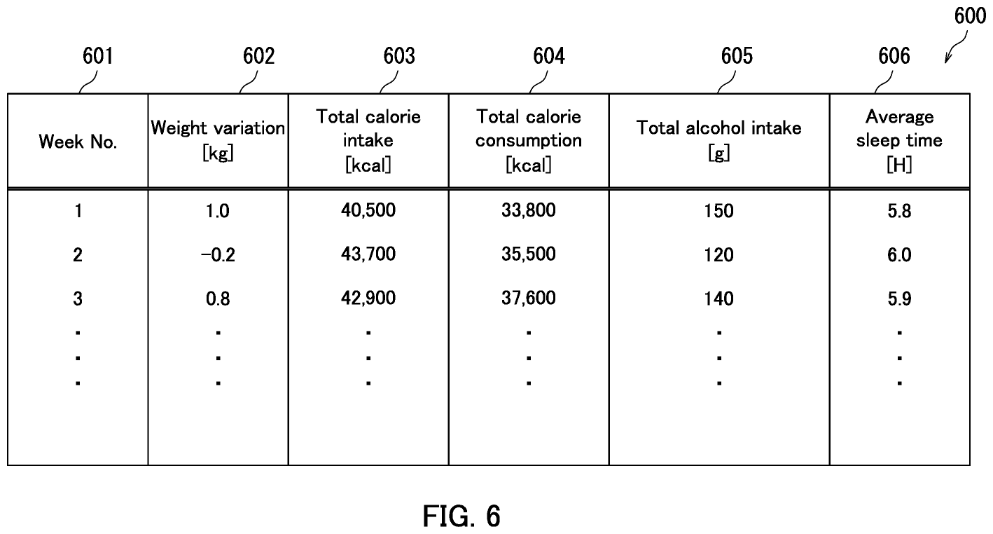

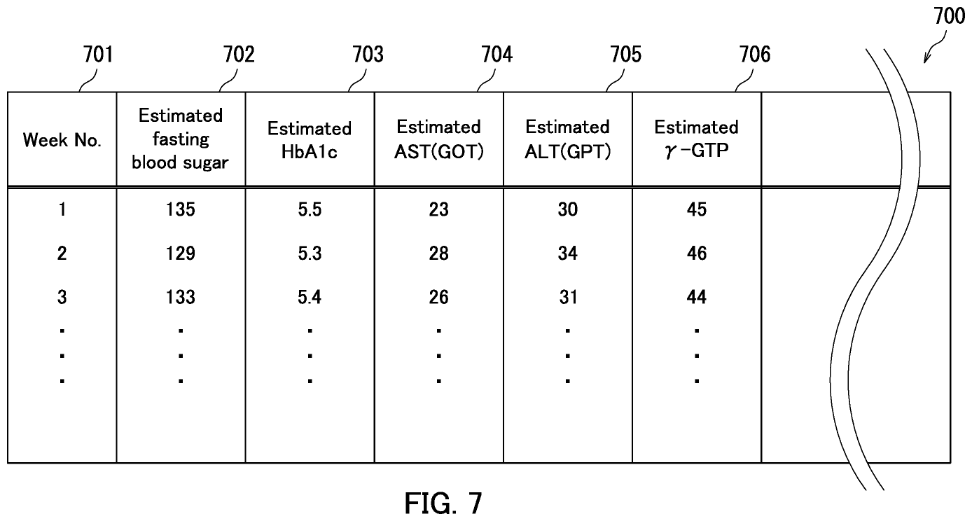

Data to be transferred between the display device 100 and the cloud server 300 will next be described with reference to FIGS. 6 and 7. FIG. 6 depicts an example of an estimation data table 600 storing the estimation data. The estimation data is transmitted from the display device 100 to the cloud server 300.

The estimation data table 600 includes a week number column (hereinafter simply called “week Nos. 601”), a weight variation column (hereinafter simply called “weight variations 602”), a total calorie intake column (hereinafter simply called “total calorie intake 603”), a total calorie consumption column (hereinafter simply called “total calorie consumption 604”), a total alcohol intake column (hereinafter simply called “total alcohol intake 605”), and an average sleep time column (hereinafter simply called “average sleep time 606”).

The week Nos. 601 represent respective numbers of weeks counted when the effect data starts being collected after the health data is captured. Each item in the weight variation (column) 602 represents an increase or decrease in body weight of the user during a corresponding week in the week Nos. 601. Each item of the total calorie intake 603 represents a total of values, of the calorie intake 503, assigned to dates of a corresponding week of the week Nos. 601. Each item of the total calorie consumption 604 represents a total of values, of the calorie consumption 504, assigned to dates of a corresponding week of the week Nos. 601. Each item of the total alcohol intake 605 represents a total of values, of the alcohol intake 505, assigned to dates of a corresponding week of the week Nos. 601. Each item of the average sleep time 606 represents an average of values, of the sleeping time 506, assigned to dates of a corresponding week of the week Nos. 601.

FIG. 7 depicts an example of an estimated result table 700 storing the estimated results. The display device 100 receives the estimated results from the cloud server 300.

The estimated result table 700 includes a week number column (hereinafter simply called “week Nos. 701”), an estimated fasting blood sugar column (hereinafter simply called “estimated fasting blood sugar 702”), an estimated HbA1c column (hereinafter simply called “estimated HbA1c 703”), an estimated AST(GOT) column (hereinafter simply called “estimated AST(GOT) 704”), an estimated ALT(GPT) column (hereinafter simply called “estimated ALT(GPT) 705”), and an estimated γ-GTP column (hereinafter simply called “estimated γ-GTP 706”).

The week Nos. 701 represent the same weeks as those of the week Nos. 601. Each item of the estimated fasting blood sugar 702 represents fasting blood sugar estimated by the cloud server 300 based on corresponding estimation data received from the display device 100. The estimation data received by the cloud server 300 from the display device 100 is hereinafter referred to as “received estimation data”. Each item of the estimated HbA1c 703, each item of the estimated AST(GOT) 704, each item of the estimated ALT(GPT) 705, and each item of the estimated γ-GTP 706 represent HbA1c, AST(GOT), ALT(GPT), and γ-GTP estimated based on corresponding received estimation data, respectively.

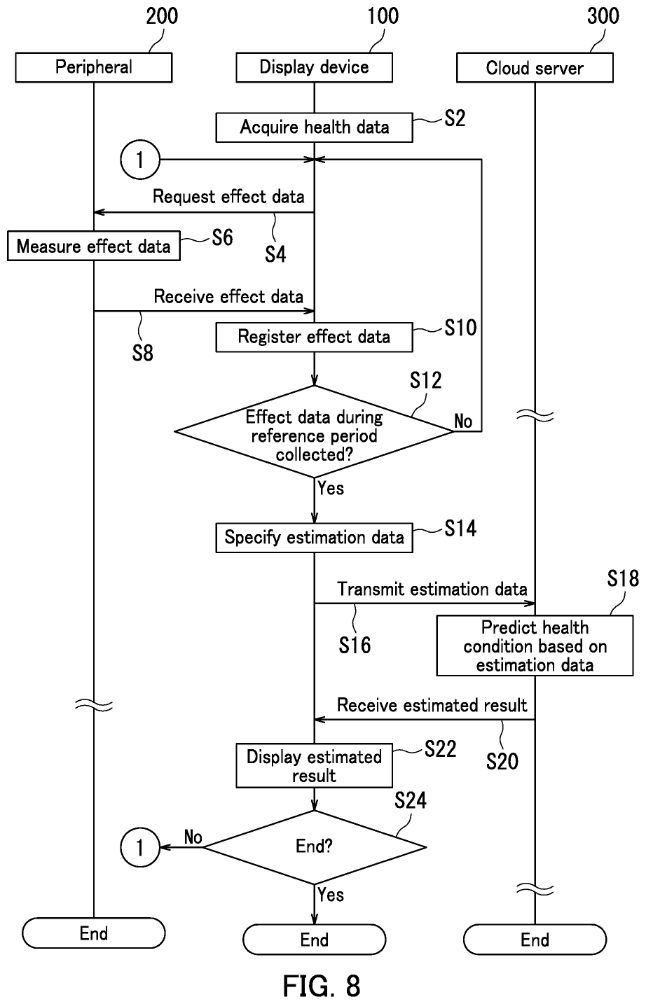

Communication in the display system 1 will next be described with reference to FIGS. 2 and 8. FIG. 8 is a communication sequence diagram of the display system 1 according to the first embodiment. The communication sequence diagram of FIG. 8 depicts a health estimation process to be executed between the peripheral 200 and the cloud server 300. The health estimation process to be executed includes Steps S2 to S24.

Step S2: the capturing section 13 photographs the medical examination result 10 to capture health data. The process then proceeds to Step S4.

Step S4: the specifying section 1512 requests the peripheral 200 to return effect data through the communication section 11. The process then proceeds to Step S6.

Step S6: the peripheral 200 measures the effect data. The process then proceeds to Step S8.

Step S8: the specifying section 1512 receives the effect data from the peripheral 200 through the communication section 11. The process then proceeds to Step S10.

Step S10: the specifying section 1512 stores, in the storage 152, the effect data received from the peripheral 200. The process then proceeds to Step S12.

Step S12: the specifying section 1512 determines whether or not respective pieces of effect data during the reference period have been collected. When the specifying section 1512 determines that the respective pieces of effect data during the reference period have been collected (Yes at Step S12), the process proceeds to Step S14. When the specifying section 1512 determines that the respective pieces of effect data during the reference period have not yet been collected (No at Step S12), the process returns to Step S4.

Step S14: the specifying section 1512 specifies estimation data. The process then proceeds to Step S16.

Step S16: the estimating section 1513 transmits the estimation data to the cloud server 300 through the communication section 11. The process then proceeds to Step S18.

Step S18: the cloud server 300 estimates health condition based the estimation data, thereby generating estimated results. The process then proceeds to Step S20.

Step S20: the processing section 151 receives the estimated results from the cloud server 300 through the communication section 11. The process then proceeds to Step S22.

Step S22: the controller 1519 controls the display section 12 so that the estimated results are displayed.

Step S24: the processing section 151 determines whether or not to complete the health estimation process. When the processing section 151 determines to complete the health estimation process (Yes at Step S24), the process ends. When the processing section 151 determines not to complete the health estimation process (No at Step S24), the process returns to Step S4.

As stated above, the display device 100 according to the present embodiment enables reporting health-related numerical values reflecting individual user specific health promotion activities.

### Second Embodiment

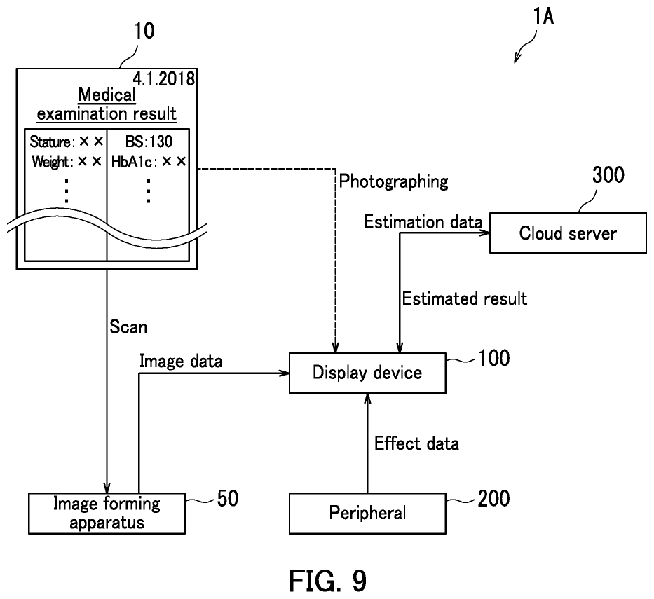

A display system 1A according to a second embodiment will next be described with reference to FIGS. 2 and 9. FIG. 9 is a schematic diagram depicting the display system 1A according to the present embodiment. In the first embodiment described with reference to FIG. 1, the display device 100 (built-in camera) photographs the medical examination result 10 to capture the health data. In contrast, the present embodiment differs from the first embodiment in that an image forming apparatus 50 scans a medical examination result 10, thereby capturing health data.

As illustrated in FIG. 9, the display system 1A includes a display device 100, a peripheral 200, a cloud server 300, and the image forming apparatus 50.

The image forming apparatus 50 scans the medical examination result 10, and generates image data representing the medical examination result 10. The image forming apparatus 50 transmits the image data to the display device 100.

The display device 100 uses an OCR function, thereby recognizing health data contained in an image of the medical examination result 10 from the image data received from the image forming apparatus 50. Note that the display device 100 causes a capturing section 13 to photograph the medical examination result 10 in order to perform display while associating the health data with an estimated image.

As stated above, the display system 1A according to the second embodiment enables a user to employ some of functions of the display device 100 as the image forming apparatus 50. This makes it possible to reduce the cost of the display device 100, and also provide a system for reporting health-related numerical values reflecting individual user specific health promotion activities.

The embodiments of the present disclosure have been described with reference to the accompany drawings (FIGS. 1 to 9). However, the present disclosure is not limited to the above embodiments and may be implemented in various manners within a scope not departing from the gist of the present disclosure (see (1) to (3) below, for example). The drawings schematically illustrate main elements of configuration to facilitate understanding thereof. Aspects of the elements of configuration illustrated in the drawings, such as thickness, length, and number, may differ in practice for the sake of convenience for drawing preparation. Furthermore, aspects of the elements of configuration illustrated in the above embodiments, such as material, shape, and dimension, are examples and are not particularly limited. The elements of configuration may be variously altered within a scope not substantially departing from the configuration of the present disclosure.

(1) Although the increase or decrease in the health data is estimated by using the cloud server 300 in the embodiments of the present disclosure, the estimation about the increase or decrease in the health data is not limited to the estimation by using the cloud server 300. When the display device 100 has an identical function to that of the cloud server 300, the display device 100 may estimate the increase or decrease in the health data.

(2) Although the embodiments of the present disclosure include an example in which the effect data is received from the peripheral 200, the present disclosure is not limited to the effect data being received from the peripheral 200. The effect data may be obtained by using big data stored in the cloud server 300.

(3) The present disclosure may also be realized as a display method including steps corresponding to the characteristic configuration means of the display device according to an aspect of the present disclosure, or as a control program including the steps. The program may also be distributed via a recording medium such as a CD-ROM or a transmission medium such as a communication network.

What is claimed is:

1. A display device, comprising:
an acquiring section configured to acquire health data on health;
a specifying section configured to specify effect data that has effect on the health data;
an estimating section configured to estimate an increase or decrease in the health data based on the effect data to obtained an estimated result; and
a display section configured to display the estimated result.

2. The display device according to claim 1, further comprising
a generating section configured to generate an estimated image representing the health data, and
a controller configured to control the display section so that display is performed with the health data associated with the estimated image.

3. The display device according to claim 2, further comprising
a capturing section configured to photograph a report including the health data to generate image data representing an image of the report, wherein
the acquiring section recognizes the health data contained in the image of the report based on the image data, and
the controller controls the display section so that the display is performed with the health data recognized by the acquiring section associated with the estimated image.

4. The display device according to claim 1, wherein
the specifying section specifies, as the effect data, at least one of values which include calorie intake every one or more days as unit of time, calorie consumption every one or more days as unit of time, alcohol intake every one or more days as unit of time, and sleeping time every one or more days as unit of time, and
the estimating section estimates the increase or decrease in the health data based on the effect data specified by the specifying section.

5. The display device according to claim 4, further comprising:
a detecting section configured to detect a signal indicating whether a light is turned on or off in a period from turning off the light to getting up; and
a computing section configured to measure sleeping time in the period based on the signal detected by the detecting section, wherein
the specifying section specifies the sleeping time every one or more days as unit of time based on the sleeping time measured by the computing section.

6. The display device according to claim 4, wherein
the specifying section specifies, as the effect data, at least one of values which include total calorie intake during a reference period, total calorie consumption during the reference period, total alcohol intake during the reference period, and average sleeping time during the reference period, and
the estimating section estimates the increase or decrease in the health data based on the effect data specified by the specifying section.

7. The display device according to claim 1, further comprising:
a determining section configured to determine whether or not a variation of the health data is greater than a first threshold but not greater than a second threshold greater than the first threshold; and
a producing section configured to produce a first message when the determining section determines that the variation of the health data is greater than the first threshold but not greater than the second threshold, wherein
the controller controls the display section so that the first message is displayed.

8. The display device according to claim 7, further comprising
a communication section configured to perform data transmission, wherein
the determining section determines whether or not the variation of the health data is greater than the second threshold,
the producing section produces a second message when the determining section determines that the variation of the health data is greater than the second threshold, and
the controller controls the communication section so that the second message is transmitted to a prescribed electronic device.

9. The display device according to claim 1, wherein
the display section is augmented reality glasses or a head mounted display.

10. A display method, comprising
acquiring health data on health,
specifying effect data that has effect on the health data,
estimating a variation in the health data based on the effect data to obtained an estimated result, and
displaying the estimated result.
